SEQUENCE LISTING (9 sequences)

SEQ ID NO: 1 - protein, 35 aa, Artificial Sequence
  Backbone of nABP386
SRLKEIANSPTQFWRMVARNTLGNHAKQSLNIEHA

SEQ ID NO: 2 - protein, 40 aa, Artificial Sequence
  Backbone of nABP486
SHHHRLKLKEIANSPTQFWRMVARNTLGNGAKQSLNIEHA

SEQ ID NO: 3 - protein, 6 aa, Artificial Sequence
  Side chain
SHHHRL

SEQ ID NO: 4 - DNA, 105 bp, Artificial Sequence
  Backbone of nABP386
tcaaggctgaaggaaatagcaaattcacctacacagttctggaggatggttgcacgaaacacactggggaatcacgcaaaacagtctttgaatatcgaacacgca

SEQ ID NO: 5 - DNA, 120 bp, Artificial Sequence
  Backbone of nABP486
tcacaccaccacaggctgaagctgaaggaaatagcaaattcacctacacagttctggaggatggttgcacgaaacacactggggaatggagcaaaacagtctttgaatatcgaacacgca

SEQ ID NO: 6 - DNA, 18 bp, Artificial Sequence
  Side chain
tcacaccaccacaggctg

SEQ ID NO: 7 - protein, 21 aa, Artificial Sequence
  Backbone of nABP786
SHHHRLKLKEIANCSPTQFKC

SEQ ID NO: 8 - DNA, 63 bp, Artificial Sequence
  Backone of nABP786
tcacaccaccacaggctgaagctgaaggaaatagcaaattgctcacctacacagttcaagtgc

SEQ ID NO: 9 - protein, 36 aa, Artificial Sequence
  nABP284
SRLKEIANSPTQFWRMVARNTLGGAKQSLNIEHARL

(19) United States
(12) Patent Application Publication
(10) Pub. No.: US 2024/0209043 A1
(43) Pub. Date: Jun. 27, 2024

(54) NON-ANTIBODY BINDING PEPTIDES AND THEIR ANALOGS DUAL-TARGETING TO PD-1 AND PD-L1, AND USES THEREOF

(71) Applicants: ORAL SUBSIDIARY SUN YAT-SEN UNIVERSITY HOSPITAL, Guangzhou, Guangdong (CN); Guangzhou Yidai Pharmaceutical Co., LTD., Guangzhou City (CN)

(72) Inventors: Hua WANG, Guangzhou, Guangdong (CN); Yilei CHEN, Guangzhou City (CN); Zhanghao WANG, Guangzhou City (CN); Lili WANG, Guangzhou City (CN); Hongxing HUANG, Guangzhou City (CN); Yan ZHANG, Guangzhou City (CN)

(21) Appl. No.: 17/908,262

(22) PCT Filed: Apr. 16, 2021
(86) PCT No.: PCT/CN2021/087714

§ 371 Date: Aug. 31, 2022

(30) Foreign Application Priority Data
Apr. 9, 2021 (CN) 202110380740.2

(51) Int. Cl. C07K 14/47; A61K 49/00; G01N 33/574

(52) U.S. Cl. CPC C07K 14/4703; A61K 49/0002; G01N 33/57492

(57) ABSTRACT

A non-antibody binding peptide or its analogs is provided, wherein the non-antibody binding peptide includes a backbone and at least one side chain linked to the backbone, wherein an amino acid sequence of the side chain is SEQ ID NO: 3, and an amino acid sequence of the backbone is at least one of SEQ ID NO: 1, SEQ ID NO: 2 or SEQ ID NO: 7. Also provided is a polynucleotide encoding the non-antibody binding peptide, a recombinant vector, and a host cell. Further provided is a preparation, including the non-antibody binding peptide or its analogs, wherein the preparation is a drug or a cell marker reagent. Further provided is a use of the non-antibody binding peptide or its analogs in preparation of a tumor diagnosis reagent, an immunoregulation drug or an anti-tumor drug.

### CROSS-REFERENCE TO RELATED APPLICATIONS

This application claims priority to PCT Application No. PCT/CN2021/087714, having a filing date of Apr. 16, 2021, which is based on Chinese Application No. 202110380740.2, having a filing date of Apr. 9, 2021, the entire contents both of which are hereby incorporated by reference.

### SEQUENCE LISTING

This application includes a separate sequence listing in compliance with the requirement of 37 C.F.R.§.§ 1.824(a)(2)-1.824 (a)(6) and 1.824 (b), submitted under the file name “0105US01_Sequence_Listing”, created on Aug. 5, 2022, having a file size of 3.02 KB, the contents of which are hereby incorporated by reference.

### FIELD OF TECHNOLOGY

The following relates to the technical field of medical biotechnology, particularly, it relates to a non-antibody binding peptide (nABP) and its analogs binding to PD-1 and PD-L1, and uses thereof.

### BACKGROUND

Programmed cell death receptor 1 (PD-1) is a membrane protein consisted of 288 amino acid residues and is expressed by several immune cells, such as T cells, B cells and myeloid cells, etc. PD-1 interacts with Programmed cell death 1-ligand 1 (PD-L1) and Programmed cell death 1-ligand 2 (PD-L2), wherein the engagement of PD-1 with its ligands upregulates E3-ubiquitin ligases, for example Cbl-b and c-Cbl, downregulate the expression of T cell antigen Receptor, and inhibit the activation of T cells and the release of cytokines. Therefore, immunoregulation targeting PD-1, plays an important role in anti-tumor, anti-infection, anti-autoimmune diseases and the survival of organ transplanting, etc.

PD-1 and its ligand negatively regulate immune responses, and it is shown by experiment that lupoid glomerulitis and dilated cardiomyopathy are more prone to occur in PD-1 knock-out mice. The binding of PD-1 to PD-L1 activates the programmed death of T cells and inhibits the secretion of interleukin 2 (IL-2) and interferon (IFN-γ). It is shown by an experimental study on viral LCMV infection, that the interaction of PD-1 with PD-L1 inhibits the activation and proliferation of virus-specific CD8 cells, while antibody drugs block the PD-1/PD-L1 binding to increase the secretion of IL-2 and rescue the negatively regulating effect on CD8 cells.

PD-1 ligand, PD-L1, is highly expressed in a variety of tumor cells. PD-L1/PD-1 pathway is an important signal pathway of tumor immune escape, and blocking the interaction of PD-1 with PD-L1 can enhance anti-tumor activity of T cells in vivo. Recently, there are 9 antibody drugs on the market. Drugs targeting PD-1, such as Nivolumab, Pembrolizumab, Cemiplimab, Toripalimab, Cindilimab, and Camrelizumab are humanized or fully human immunoglobulin G 4 (Ig G4) antibodies; and drugs targeting PD-L1, such as Atezolizumab, Avelumab and Durvalumab are humanized immunoglobulin G 1 (IgG1) antibodies or are fully human immunoglobulin G 1 (IgG1) antibodies. There is no drug dual-targeting at PD-1/PD-L1 on the market.

Affinity is one of the important factors that affect the efficacy, dosage, and side effects of molecular targeted drugs, and is jointly determined by the binding region, binding area and the adaption of the concave-convex binding interactions in stereo-configuration. The affinity is characterized by dissociation constant (Kd), and the smaller Kd-value is, the larger affinity is. A main binding site for Nivolumab is located in a ‘N-terminal loop’ structure of PD-1, binding area is about 1487 Å2, Kd-value is 1.45 nmol/L; the main binding site for Pembrolizumab is located in a C′D loop structure, binding area is about 2126 Å2, Kd-value is 27 pmol/L; Kd-value for Cemiplimab is 6.11 nmol/L; Toripalimab and Cindilimab mainly bind to FG loop, their binding areas are 2011 Å2 and 322 Å2, respectively, Kd-value is 0.324 nmol/L and 0.25 nmol/L; and Camrelizumab binds to CC′ loop and FG loop, binding area is 2520 Å2, Kd-value is 3.31 nmol/L. Atezolizumab binds to an antiparallel-beta-pleated sheet by three CDR rings from heavy chains and one CDR ring from a light chain, that is, it interacts with residues in BC loop, CC′ loop, C′C″ loop and FG loop, binding area is 2106 Å2, Kd-value is 400 pmol/l; the region of binding epitope for Avelumab is consisted of C chain, C′ chain, F chain, G chain and CC′ loop of PD-L1, binding area is 1856 Å2, Kd-value is 42.1 pmol/L; and the binding region of Durvalumab is consisted of CC′ loop and N′-terminal of PD-L1, binding area is 1624 Å2, Kd-value is 667 pmol/L.

Chinese patent ZL 201710324664.7 discloses a non-antibody binding protein (PD-1-nABP284) for binding to PD-1 receptor, which can specifically bind to PD-1 receptor. It was shown by experiments in vitro that nABP284 can block the binding of PD-L1 ligand to PD-1, enhance the secretion of interleukin 2 (IL-2) and tumor cytotoxicity of T lymphocytes, and thus inhibit tumor growth in mice. Compared with a monoclonal antibody, non-antibody binding peptides are synthesized easily and their qualities are easily controlled. And they rarely induce immunogenicity, have better tumor tissue targeting effect and permeability with a low manufacture cost, but have a low affinity for PD-1 (receptor). There is no experiment shown that nABP284 can bind to the PD-1 ligand, PD-L1.

### SUMMARY

An aspect relates to a non-antibody binding peptide (nABP) for a PD-1 receptor, which have a significantly enhanced affinity for PD-1 and a significantly enhanced blocking effect on PD-L1, and improve the secretion of interleukin 2 and cytoxicity of T lymphocytes to cancer cells.

Another aspect relates to provide a use of the non-antibody binding peptide.

An aspect relates to a non-antibody binding peptide or its analogs for a PD-1 receptor, wherein the non-antibody binding peptide comprises a backbone and at least one side chain linked to the backbone, wherein an amino acid sequence of the side chain is SEQ ID NO: 3, and an amino acid sequence of the backbone is at least one of SEQ ID NO: 1, SEQ ID NO: 2 or SEQ ID NO: 7. A leucine (Leu, or L) in the at least one (such as one, two, or three, etc) side chain (SHHHRL) binds to a lysine (Lys, or K) in the backbone, thus the side chain binds to the backbone. In an embodiment, the backbone and the side chain of the non-antibody binding peptide may be SEQ ID NO: 1 and SEQ ID NO: 3, respectively. And this non-antibody binding peptide can be named as nABP386, wherein an L in the side chain is linked to the position 27^{th} K in the backbone. Alternatively, the backbone and the side chain of the non-antibody binding peptide are SEQ ID NO: 2 and SEQ ID NO: 3, respectively. And this non-antibody binding peptide can be named as nABP486, wherein an L in the side chain is linked to the position 7^{th} K in the backbone. Alternatively, the backbone and the side chain of the non-antibody binding protein are SEQ ID NO: 7 and SEQ ID NO: 3, respectively. And this non-antibody binding protein can be named as nABP786, wherein an L in the side chain is linked to the position 7^{th} K in the backbone.

The term “analogs”, as used herein refers to peptides that differ in amino acid sequence at one or more amino acid positions when the sequences are aligned.

The term “polynucleotide” refers to a DNA, RNA, chimeric DNA/RNA molecule, or a DNA strand hybridized to an RNA strand. A “polynucleotide” may have one or more modified bases.

The term “Kd”, as used herein refers to a dissociation constant of a specific antibody-antigen interaction, which is used to describe the binding affinity of an antibody to an antigen.

Another aspect relates to a polynucleotide, which encodes the non-antibody binding peptide.

In an embodiment, a nucleotide sequence encoding the backbone of the non-antibody binding peptide is SEQ ID NO: 4, and a nucleotide sequence encoding the side chain of the non-antibody binding peptide is SEQ ID NO: 6.

In an embodiment, a nucleotide sequence encoding the backbone of the non-antibody binding peptide is SEQ ID NO: 5, and a nucleotide sequence encoding the side chain of the non-antibody binding peptide is SEQ ID NO: 6.

In an embodiment, a nucleotide sequence encoding the backbone of the non-antibody binding peptide is SEQ ID NO: 8, and a nucleotide sequence encoding the side chain of the non-antibody binding peptide is SEQ ID NO: 6.

Another aspect relates to a recombinant vector comprising the polynucleotide.

Another aspect relates to a host cell comprising the recombinant vector.

Another aspect relates to a preparation comprising the non-antibody binding peptide or its analogs, wherein the preparation is a drug or a cell marker reagent.

In an embodiment, when the preparation is a drug, it further comprises a pharmaceutically acceptable excipient or carrier; when the preparation is a cell marker reagent, it further comprises an acceptable carrier, or excipient for molecular markers.

In an embodiment, the preparation comprises the non-antibody binding peptide or its analogs, linked to a compound comprising at least one of a pharmaceutical molecule or a detective marker, wherein a backbone of the non-antibody binding peptide is at least one of SEQ ID NO: 1, SEQ ID NO: 2, or SEQ ID NO: 7, and a side chain of the non-antibody binding peptide is SEQ ID NO: 3.

In an embodiment, the pharmaceutical molecule has an ability of preventing and/or treating a tumor and an autoimmune disease activated by an immunity checkpoint of PD-1. In an embodiment, the tumor is a tumor with up-regulated PD-L1 expression, and includes but is not limited to melanoma, non-small cell lung cancer, breast cancer, soft tissue sarcoma, head-neck neoplasm.

In an embodiment, the detective marker comprises one or more selected from the group of fluorescent reagent, nuclide reagent, or radioactive reagent.

Another aspect relates to a use of a non-antibody binding peptide or its analogs in preparation of diagnosis reagent of a tumor or an immune disease. In an embodiment, the tumor or immune disease is a disease with PD-L1 and PD-1 abnormal expression.

Another aspect relates to a use of a non-antibody binding peptide or its analogs for binding a marker or preparation to PD-L1 and PD-1 positive-cells.

Another aspect relates to a use of a non-antibody binding peptide or its analogs in preparation of a preparation for labeling, identifying, enriching, sorting or purifying PD-1 positive-cells. In an embodiment, PD-1 is derived from total cell proteins, cell secreted protein, or live cell surface. The PD-1 positive-cell includes but is not limited to T cells, NK cells, or leukemia cells.

Another aspect relates to a use of a non-antibody binding peptide or its analogs in preparation of an immunoregulation drug.

Another aspect relates to a use of a non-antibody binding peptide or its analogs in preparation of immunoregulation drug for treating a disease with increasing secretion of PD-L1 in vivo, alternatively, a use of a non-antibody binding peptide or its analogs in preparation of a preparation for enhancing a tumor killing effect of cultured immune cells in vitro.

Another aspect relates to a use of a non-antibody binding peptide or its analogs in preparation of an anti-tumor drug. In an embodiment, the tumor is a tumor with up-regulated PD-L1 expression, and includes but is not limited to melanoma, non-small cell lung cancer, breast cancer, soft tissue sarcoma, head-neck neoplasm. In an embodiment, it is another aspect of the present disclosure to provide a use of a non-antibody binding peptide or its analogs for acting as a blocking agent of PD-1/PD-L1 pathway.

It is shown in a vitro experiment that the binding affinity (Kd-value) of nABP486 to PD-1 is 11.9 nmol/L, while the Kd-value of nABP386 to PD-1 is 75.1 nM, which is significantly larger than that of nABP284, 11.8 μM.

It is shown in a further experiment that the binding affinity (Kd-value) of nABP486 to PD-L1 is 54.2 nmol/L, indicating that the nABP486 has a dual-targeting binding effect on PD-1 and PD-L1.

It is shown in a further experiment that the binding affinity (Kd-value) of nABP786 to PD-1 is 5.84 nmol/L, while the Kd-value of nABP786 to PD-L1 is 16.7 nmol/L, indicating that nABP786 has a dual-targeting binding effect on PD-1 and PD-L1, and has a higher binding affinity to a targeted molecular than nABP486.

It is found by a research that nABP386 or nABP486 can specifically bind to PD-1 on the surface of lymphocytes, while the affinity of nABP386 or nABP486 to PD-1 is higher than that of nABP284 to PD-1 in the prior art.

It is found by a research that nABP386 or nABP486 competes with PD-L1 ligand to bind to a PD-1 receptor on the surface of Jurkat T in tumor cell lines, and plays a role similar to PD-1 targeted monoclonal antibody drug, having a competitive binding effect, and therefore has a better blocking effect than nABP284 in the prior art.

It is found by a research that the binding of nABP386 or nABP486 to PD-1 on the surface of lymphocytes can reverse lymphocytes that are inhibited by PD-L1 on the surface of tongue cancer cells and recover the secretion of IL-2, indicating that nABP386 and nABP486 play a role similar to PD-1 targeted monoclonal antibody drug having a humoral immunomodulatory effects, and therefore have a better immunomodulatory effect than nABP284 in the prior art.

It is found by a research that nABP386 or nABP486 has non-toxic and side effects on peripheral blood derived lymphocytes, tongue cells and Jurkat T cells, and the addition of nABP386 or nABP486 to a co-culture solution of lymphocytes and tongue cancer cell line CAL27 can enhance the tumor killing effect of lymphocytes on tumor cells, which is similar to anti-tumor effect of PD-1 targeted monoclonal antibody drug, and therefore nABP386 and nABP486 have a better anti-tumor effect, compared with nABP284 in the prior art.

Compared with therapeutic antibodies, the non-antibody binding peptides nABP486 and nABP786 have an dual-targeting binding effect on PD-1/PD-L1, and the non-antibody binding peptides nABP386, nABP486 and nABP786 are artificially synthesized, and have lower immunogenicity and higher stable in body, better permeability and increased accumulation in tumors with a lower manufacture cost.

In conclusion, the non-antibody binding peptides nABP386, nABP486 and nABP786 of the present disclosure have many benefits. Firstly, the non-antibody binding peptides have small molecule weight and good permeability; secondly, they can be artificially synthesized with low cost, non-animal origin, and high safety; thirdly, they can well bind to specific molecules and have an effect similar, or even superior to monoclonal antibody; further, the non-antibody binding peptides nABP386, nABP486 and nABP786 of the present disclosure have an affinity close to the monoclonal antibody, which improves the low affinity of non-antibody binding peptide nABP284 to a PD-1 receptor (disclosed in Chinese patent ZL201710324664.7); and finally and importantly, the non-antibody binding peptides nABP486 and nABP786 have a dual-target binding effect on PD-1 and PD-L1, which is superior to monoclonal antibodies in single-target inhibition.

### BRIEF DESCRIPTION

Some of the embodiments will be described in detail, with reference to the following figures, wherein like designations denote like members, wherein:

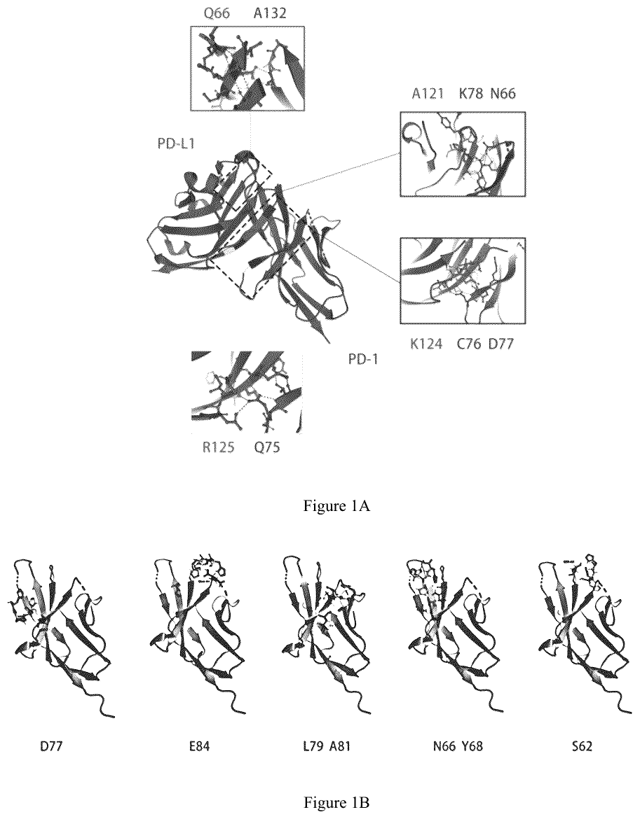

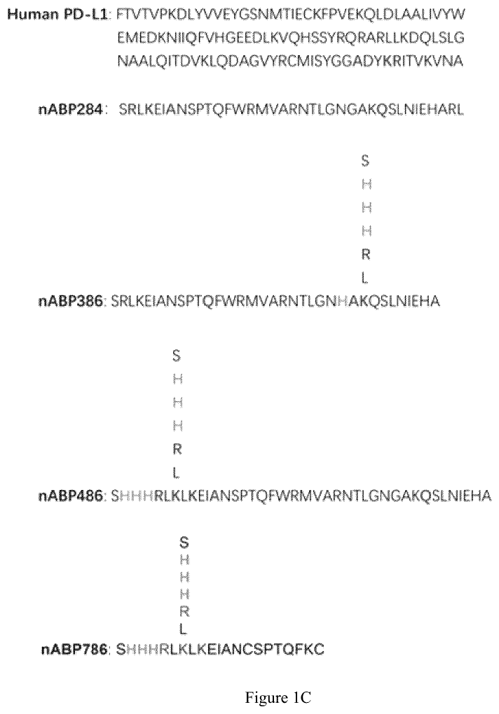

FIG. 1A depicts two main binding regions of PD-1 and PD-L1, wherein Region 1 comprises amino acids residues A121, K124, R125, etc, and Region 2 comprises amino acids residues Q66, etc;

FIG. 1B depicts a modelled binding site of a short chain comprising histidine that can enhance the affinity of nABP284 to human PD-1 through computer modeling; and

FIG. 1C depicts amino acid sequences comprising human PD-L1, nABP284, nABP386, and nABP486 and homologous comparison thereof.

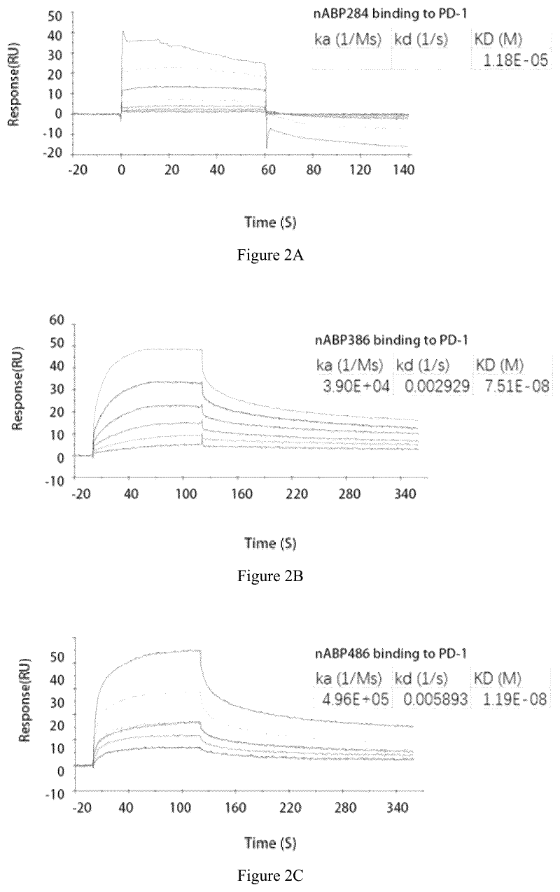

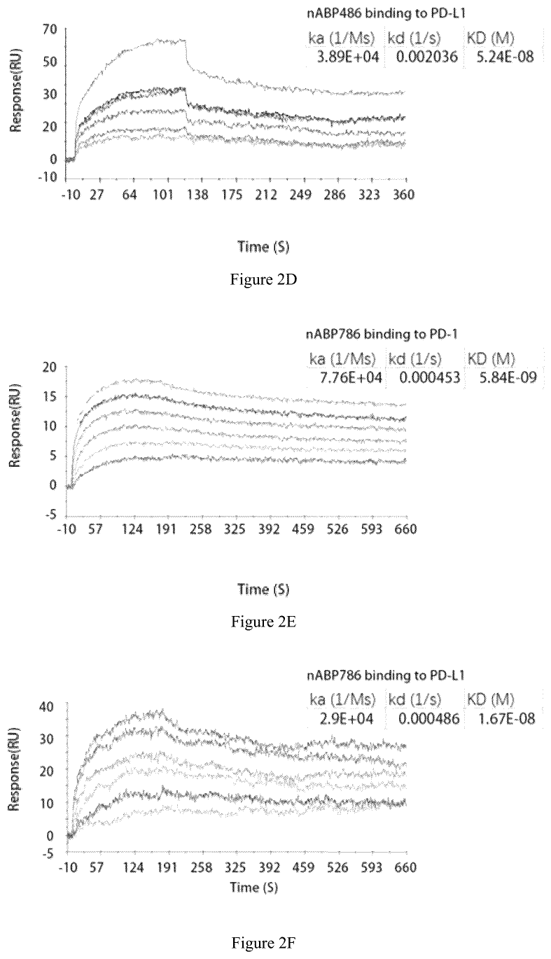

FIG. 2A depicts test results of binding affinity of nABP284 to PD-1 measured by surface plasmon resonance (SPR);

FIG. 2B depicts test results of binding affinity of nABP386 to PD-1 measured by SPR;

FIG. 2C depicts test results of binding affinity of nABP486 to PD-1 measured by SPR;

FIG. 2D depicts test results of binding affinity of nABP486 to PD-L1 measured by SPR;

FIG. 2E depicts test results of binding affinity of nABP786 to PD-1 measured by SPR;

FIG. 2F depicts test results of binding affinity of nABP786 to PD-L1 measured by SPR;

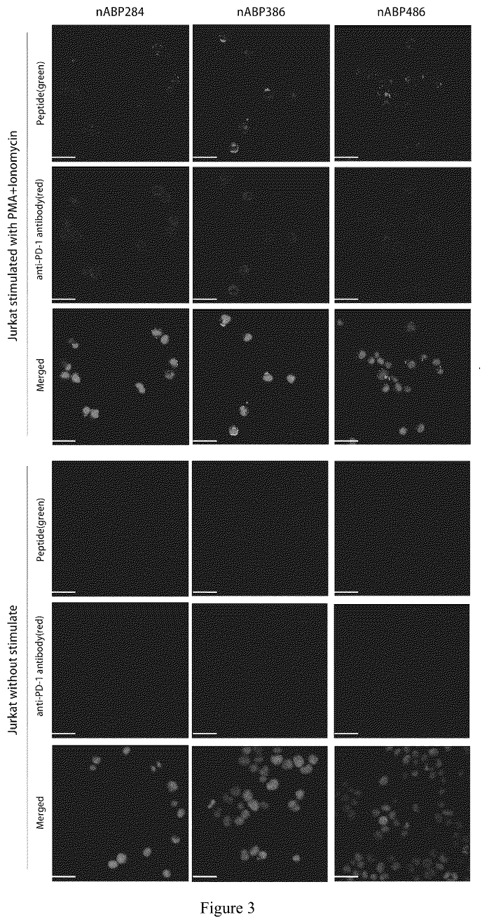

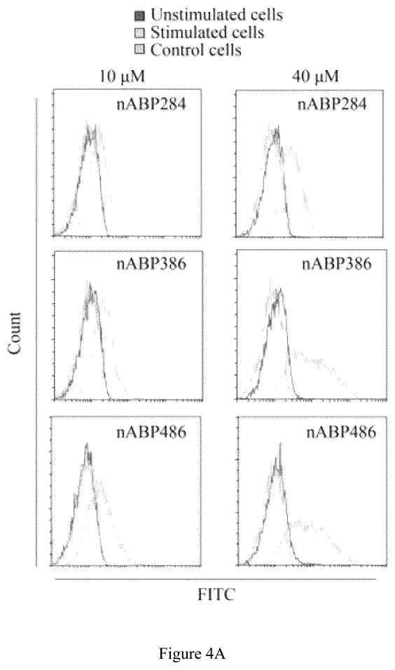

FIG. 3 depicts immunofluorescence results of nABP284, nABP386, or nABP486 specifically binding to PD-1 on the surface of Jurkat T cells, respectively;

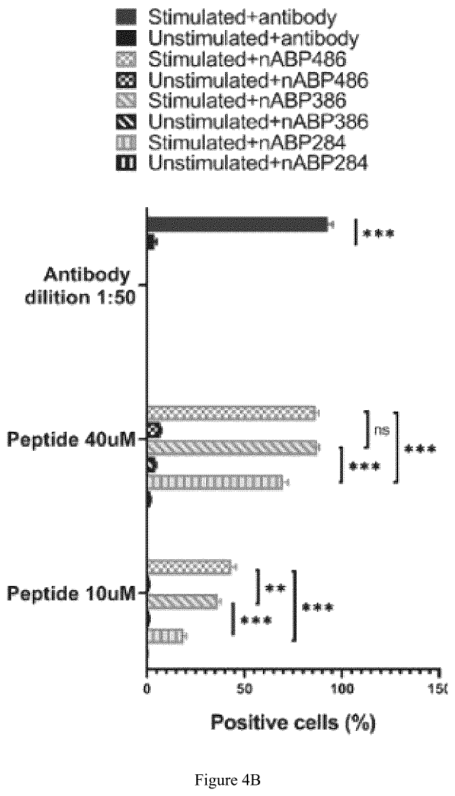

FIG. 4A depicts immunofluorescence analysis of cell counts using FITC flow cytometry;

FIG. 4B depicts binding rates of nABP284, nABP386, or nABP486 in a variety of concentrations binding to PD-1 on the surface of Jurkat T cells, respectively;

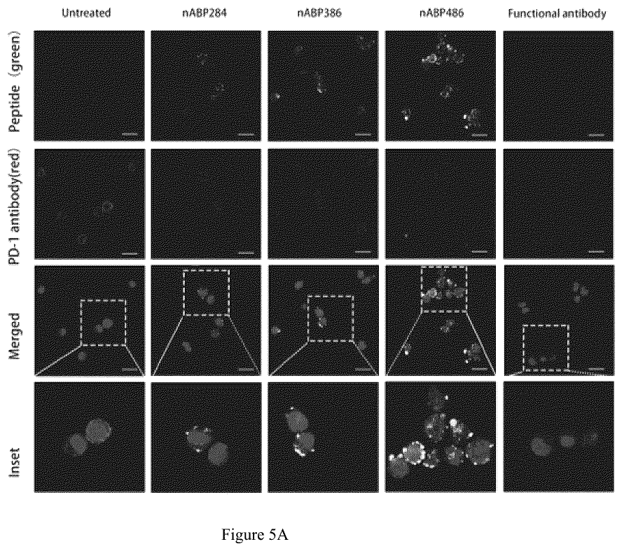

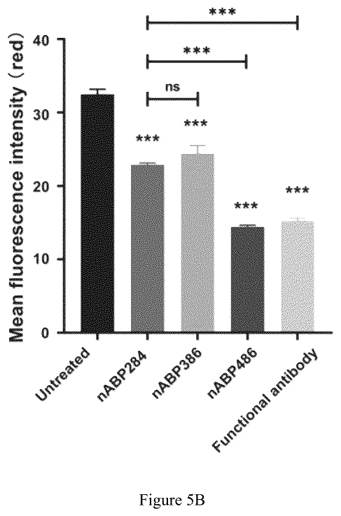

FIG. 5A depicts immunofluorescence results showing abilities of nABP284, nABP386, or nABP486 competitively binding to PD-1 on the surface of cells;

FIG. 5B depicts the quantitative results of the mean fluorescence intensity;

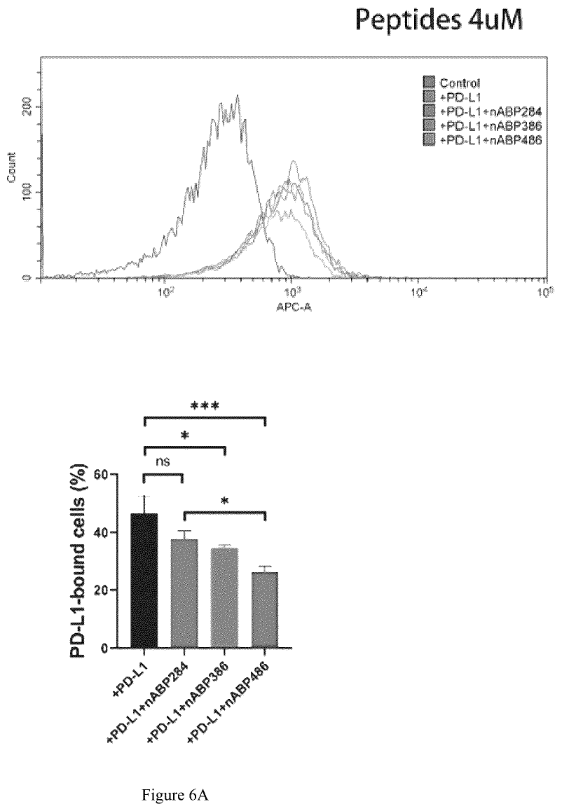

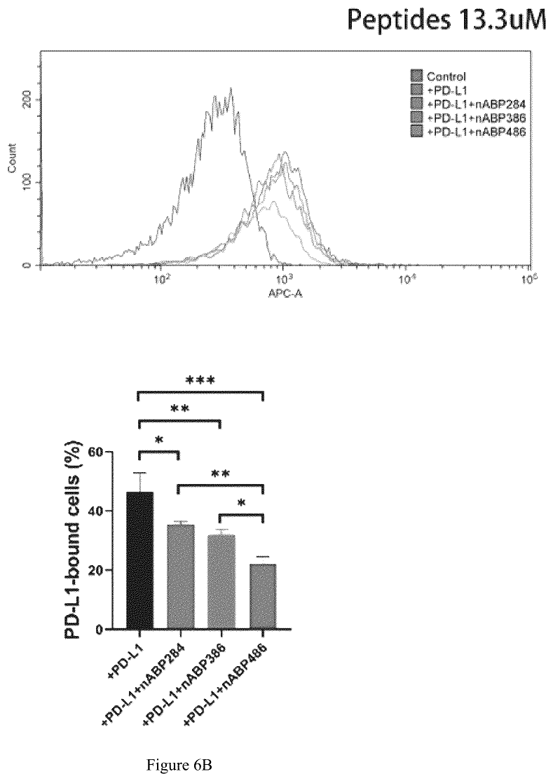

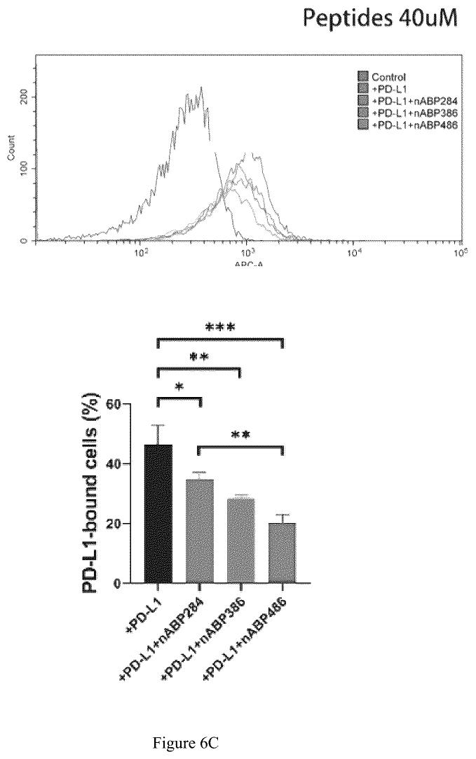

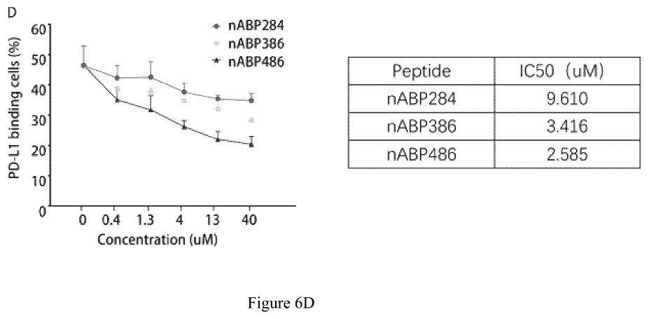

FIG. 6A depicts neutralizing abilities of nABP284, nABP386, or nABP486 at 4 μM to recombinant Human PD-L1 protein;

FIG. 6B depicts neutralizing abilities of nABP284, nABP386, or nABP486 at 13.3 μM to recombinant Human PD-L1 protein;

FIG. 6C depicts neutralizing abilities of nABP284, nABP386, or nABP486 at 40 μM to recombinant Human PD-L1 protein;

FIG. 6D depicts binding rates of nABP284, nABP386, or nABP486 at a variety of concentrations to recombinant Human PD-L1 protein and inhibitory activity of nABP284, nABP386, or nABP486 to recombinant Human PD-L1 protein;

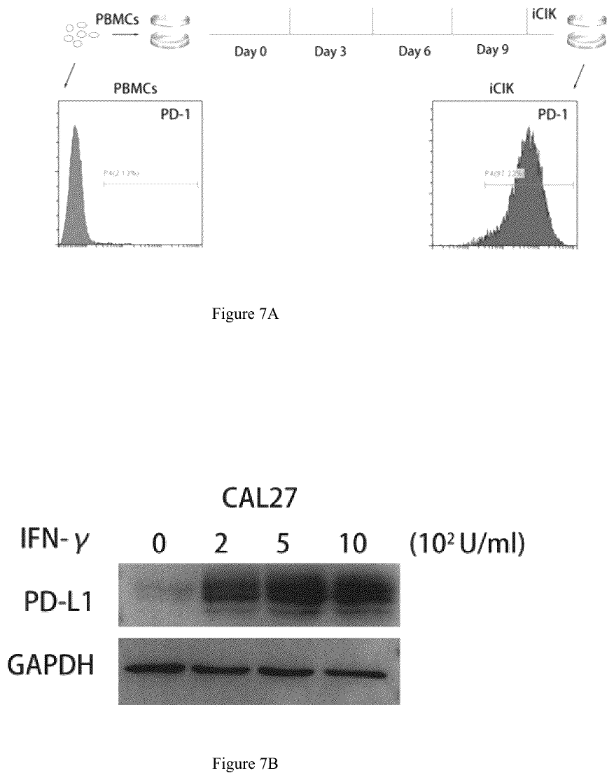

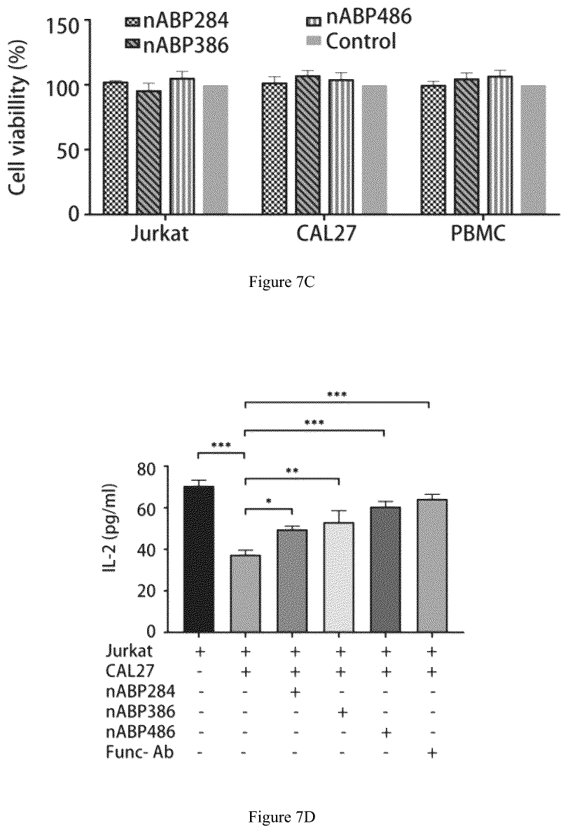

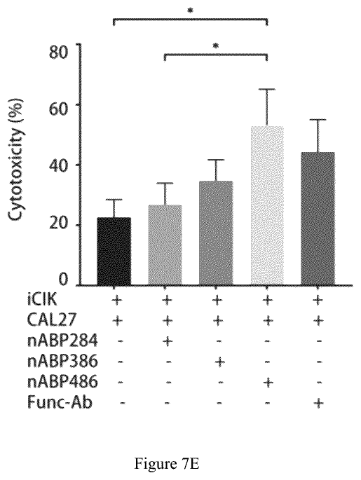

FIG. 7A depicts a method of culturing and inducing human peripheral blood mononuclear cells (PBMCs) in vitro (PBMCs cultured and induced in vitro are known as improving cytokine-induced killer cells, iCIKs) and shows the expression rates of PD-1 on the surface of PBMCs and iCIKs, respectively;

FIG. 7B depicts the expression quantity of PD-L1 on the surface of Cal27 cells after a 48-hour stimulation with different concentrations of IFN-γ;

FIG. 7C depicts cytotoxicity results of nABP284, nABP386, or nABP486 on Jurkat T cells, Cal 27 cells, and iCIKs;

FIG. 7D depicts the detection results of an increased secretion of interleukin by blocking PD-1/PD-L1 binding via nABP284, nABP386, or nABP486; and

FIG. 7E depicts the detection results of an increased tumor killing effect by blocking PD-1/PD-L1 binding via nABP486.

### DETAILED DESCRIPTION

The present disclosure will be further explained with reference to the following examples hereinafter. It should be understood that the examples are used for illustrating the present discloses only but do not limit the scope of the present disclosure.

Main research methods used in the examples comprise modeling with AutoDock and Pymol software, confocal microscope observation for immunofluorescence assay of co-labeling non-antibody binding polypeptide and antibody, surface plasma resonance, flow cytometry, cytotoxicity assay, western blot, enzyme-linked immunosorbent assay and so on. Unless otherwise specified, the experimental operations are routine experimental operations learned by the skilled person in the art. The reagents used are commercially available, unless otherwise specified.

Some reagents used in the examples are illustrated as follows:

Recombinant Human PD-1 protein (PD1-H5221, Acro Biosystems), Recombinant Human PD-L1 protein (PD1-H82F3, Acro Biosystems), Human PD-1 antibody (ab52587, abcam), Human PD-1-Fc-antibody (130-120-389, Miltenyi Biotec), APC Streptavidin (405207, Biolegend), goat anti-mouse IgG secondary antibody (ab150115, abcam), RPMI1640 (R6504, Sigma-Aldrich), DMEM/F12 (D0697, Sigma-Aldrich), Fetal Bovine Serum (FBS, 10099-141, Gibco), human recombinant interleukin-2 (200-02, PEPROTECH), Human recombinant interferon gamma (300-02, PEPROTECH), Phorbol 12-myristate 13-acetate (1652981, PeproTech), Ionomycin Calcium Salt (5608212, PeproTech), PD-1 functional monoclonal antibody (16-9989-82, eBioscience Thermo Fisher Scientific), Anti-CD3 monoclonal antibody (T210, TAKARA).

### 1. Obtaining and Culturing Cells

Human T-cell acute lymphoblastic leukemia cell line (Jurkat T cells) and Human tongue squamous cancer cell line (Cal27 cells) were commercially available from the National Collection of Authenticated Cell Cultures (Shanghai, China). Jurkat T cells were cultured in RPMI1640 medium with 10% FBS, and Cal27 cells were cultured in DMEM/F12 medium with 10% FBS, and human peripheral blood mononuclear cells (PBMCs) were obtained from peripheral blood of healthy adult donor through a method of density gradient centrifugation, cultured in RPMI1640 medium with 10% FBS, 2000 U/ml of INF-γ, and 10 ng/ml of IL-2.

### 2. Design and Synthesis of New Peptides

The critical binding region of Human PD-L1/Human PD-1 was searched and amino acid residues sequence of PD-L1 in the critical region were compared with an amino acid sequence of nABP284 (SEQ ID NO: 9, SRLKEIANSPTQF WRMVARNTLGGAKQSLNIEHARL), which was screened by Phage display technology, wherein 2 RLKEIA 7 and 28 Q in nABP284 were identified as a critically specific binding region. It is a fact that a crack between Y 68 and E 136 near the critical binding region of PD-1 just provided enough room to accommodate a ring structure on a side chain of amino acids, and histidine had an imidazole ring. In addition, it seems that the addition of histidine can enhance the binding affinity of nABP284 to PD-1. Therefore, histidine was added near the critical binding region in nABP284 to form a side chain and obtain nABP386 and nABP486. For example, for nABP386, leucine (L) in the side chain was linked to the position 27^{th} lysine (K) in the backbone; for nABP486 and nABP786, leucine (L) in the side chain was linked to the position 7^{th} lysine (K) in the backbone.

New peptides were synthesized by China Peptides Co., Ltd through a solid-phase synthesis method and their quality was tested by high performance liquid chromatography and electrospray mass spectrometry.

### 3. Affinity Assay of New Peptides

The affinity of the peptides was tested with Biocore T100 through surface plasmon resonance (SPR) technique. Cross-linking agent EDC/NHS was used to esterify the surface of a chip (CM5) at pH 4.5; recombinant Human PD-1 protein or PD-L1 protein was coupled into the chip in a concentration of 5 ug/ml; excess active carboxyl groups on the chip were blocked with 1M ethanolamine hydrochloride (pH 8.5); an channel un-coated with recombinant Human PD-1 protein was used as a reference channel; all SPR signals were calibrated by deducting a corresponding value of the reference channel; a peptide stock solution was diluted in a series of running buffer (pH 7.4, 100 mM Tris, 150 nM NaCl, 0.05% Tween-20); the affinity of nABP284 was analyzed by one-round SPR measurement in seven concentrations (20 μM, 10 μM, 5 μM, 2.5 μM, 1.25 μM, 0.625 μM, and 0.3125 μM), and the affinities of nABP386, nABP486 and nABP786 were analyzed by one-round SPR measurement in seven concentrations (2 μM, 1 μM, 0.5 μM, 0.25 μM, 0.125 μM, 0.0625 μM, and 0.03125 μM), respectively; after association and dissociation in each round, the chip CM5 was regenerated with 10 mM glycine-HCl (pH2.5). Finally, the affinities (Kd-value) of the peptides were calculated by fitting curves in the sensor diagram in Biacore software.

### 4. Immunofluorescence Analysis and Flow Cytometry Analysis of Specific Binding on New Peptides

Experiments were divided into an experimental group and a control group. In the experimental group, Jurkat T cells were stimulated for 24 hours with 50 ng/ml of Phorbol 12-myristate 13-acetate (PMA) and 1 ug/ml of Ionomycin Calcium Salt (Ionomycin) to express PD-1, while in the control group, Jurkat T cells were not stimulated and thus hardly expressed any PD-1. For immunofluorescence analysis, Jurkat T cells (1×10⁶) were obtained and blocked with 2% BSA for 1 hour at room temperature to decrease specific binding. After blocking, the cells were collected and incubated with 10 μM of peptides stained with FITC (green fluorescence) for 1 hour at 37° C. PBS was used for washing the cells three times and 4% paraformaldehyde was used for immobilization. Then, PBS was used for washing three times again, and the precipitated cells were resuspended with PD-1 antibodies (ab52587) diluted at a ratio of 1:100 and incubated overnight at 4° C. Then, PBS was used for washing three times again, and goat anti-mouse IgG polyclonal antibodies (ab150115) were used for staining the cells, followed by incubating in dark for 1 hour at 4° C. PBS was used for washing three times again, 4′,6-diamidino-2-phenylindole (DAPI) at a ratio of 1:1000 was used for staining cell nucleus. PBS was used for washing three times again and anti-fade mounting medium was used for preserving fluorescence. Further, fluorescence confocal microscope images were analyzed by using Image J software to evaluate the ability of polypeptides to compete the binding sites of PD-1. For the flow cytometry analysis, Jurkat T cells (2×10⁵) were obtained and blocked with 2% BSA for 1 hour at room temperature to decrease non-specific binding. Subsequently, the cells were incubated with 10 μM or 40 μM of peptides stained with FITC (green fluorescence) for 1 hour at 4° C. PBS was used for washing three times, and finally 500 ul of PBS was used for resuspending the cells, which were then transferred to flow tubes and analyzed by flow cytometry (CytoFLEX, BECK MANCOULTER, America).

### 5. Neutralization of New Peptides

The Jurkat T cells were divided into two groups, an experimental group and a control group, wherein in the experimental group, 50 ng/ml of Phorbol 12-myristate 13-acetate (PAM) and 1 ug/ml of Ionomycin Calcium Salt (Ionomycin) were used to stimulate the cells, and in the control group, Jurkat T cells were unstimulated. The cells (5×10⁵) were collected in each tube, and washed once with FACS buffer (PBS with 2% BSA). Then recombinant Human PD-L1 protein (PD1-H82F3, Acro Biosystems) was diluted to 4 ug/ml with FACS, and PD-1 binding peptides were diluted with FACS to a series of concentrations of 0.4 μM, 1.33 μM, 4 μM, 13.3 μM, and 40 μM. Subsequently, the two working solutions (PD-L1 protein diluent and peptide diluent) were mixed in an equal volume and 100 ul of mixed solutions at different concentrations were added to the centrifuge tubes together with the cells, incubated for 1 hour at 4° C. FACS buffer was used for washing the cells three times, and 0.12 ug/ml of APC Streptavidin (405207, Biolegend) was added to each tube and the incubation was performed for 1 hour at 4° C., and then the cells were washed three times with FACS buffer again, and 500 μl of PBS was added to resuspend the cells, which were further transferred to flow tubes and analyzed by flow cytometry.

### 6. Cytoxicity Assay of New Peptides

Cytotoxicity assay was performed and measured with Cell Counting Kit-8 (CCK-8, Dujindo, Japan). Jurkat cells (each well with 4000 cells), PBMCs (each well with 4000 cells) and Cal27 cells (each well with 2000 cells) were added into a 96-well plate and cultured for 24 hours, respectively. nABP284, nABP386 and nABP486 in a concentration gradient of 1 μM, 2 μM, 3 μM, 4 μM, 8 μM, 16 μM, 32 μM and 64 μM were added into the medium and the cells were cultured for 24 hours in a cell incubator. After that, 10 μl of CCK-8 solution was added to each well and the cells were incubated for 2 hours. VictorX5 Multilabel plate reader (PerkinElmer, Singapore) was used for measuring absorbance of each well at 450 nm. Cell viability (%) was calculated by the following formula:

Cell viability (%)=[(Sample A−Background A)/(Control A−Blank A)×100%.
Wherein Sample A denotes the absorbance of the experimental well (including cells, CCK-8 solution, medium and peptide diluent)
Background A denotes the absorbance of the background hole (including CCK-8 solution, medium, and peptide diluent without cells);
Control A denotes the absorbance of the control hole (including cells, CCK-8 solution and medium without peptide diluent); and
Blank A denotes the absorbance of the blank hole (including CCK-8 solution and medium without cells and peptide diluent).

### 7. Co-culture of Stimulated Jurkat T Cells and Stimulated Cal27 Cells, and Cell Viability Analysis of Jurkat T Cells

50 ng/ml of Phorbol 12-myristate 13-acetate (PMA) and 1 ug/ml of Ionomycin Calcium Salt (Ionomycin) were used for stimulating Jurkat T cells for 24 hours, and 500 U/ml of IFN-γ was used for stimulating CAL27 cells for 48 hours. The stimulated Jurkat T cells were incubated with 10 μM of nABP284, 10 μM of nABP386, 10 μM of nABP486, and 2 ug/ml of PD-1 functional monoclonal antibody (J116, eBioscience|ThermoFisher Science) for 1 hour in advance at 37° C., respectively, and the stimulated CAL27 cells were added to the 96-well plate at 1×10⁴ cells per well. When CAL27 cells were immobilized, a supernate was removed. The Jurkat T cells at a ratio of 4 to 1 and 200 ul of medium were added to each well. After these cells were co-cultured for 24 hours, a supernate was collected and analyzed by ELISA kit (88-7025-86, Thermo Fisher Scientific) to measure the amount of IL-2 in the supernate.

8. Impact of Tumor Killing Efficiency of New Peptides on Improving Cytokine-Induced Killer (iCIK)

Human peripheral blood and PBS are mixed at a ratio of 1 to 1, and then the mixture was added to the surface of Ficoll solution in an equal volume and the mixture and Ficoll were kept layered. Density gradient centrifugation (400 g, 20 min) was used for separating components of the peripheral blood. 10 ug/ml of Anti-CD3 monoclonal antibody (T210, TAKARA) was coated on cell culture dishes to cover the mixture at 4° C. overnight. The separated PBMCs were added to the coated dishes in which RPMI1640 medium supplemented with 10% of fetal calf serum, 2000 U/ml of IFN-γ, and 10 ng/ml of IL-2 was added and was changed every three days. After a ten-day culture, the stimulated PBMCs were known as improving cytokine-induced killer cells (iCIKs).

Analysis of iCIKs-mediated tumor killing efficiency.

500 U/ml of IFN-γ was used for stimulating Cal27 cells for 48 hours. The iCIKs were incubated with 10 uM of nABP284, 10 μM of nABP386, 10 μM of nABP486, and 2 ug/ml of PD-1 functional monoclonal antibody (J116, eBioscience|ThermoFisher Science) for 1 hour at 37° C., respectively, and the stimulated CAL27 cells were added to the 96-well plate at 1×10⁴ cells per well. When CAL27 cells were immobilized, a supernate was removed. The iCIKs at a ratio of 10 to 1 and 100 ul of medium were added to each well. The 96-well plate was centrifuged at 250 g for 4 minutes to make iCIKs completely contacted with Cal27 cells. After these cells were co-cultured for 6 hours, a Cyto Tox Non-Radioactive Cytotoxicity Assay kit was used for detecting the level of lactic dehydrogenase (LDH) in the medium and VictorX5 multilabel plate reader (PerkinElmer, Singapore) was used for measuring absorbance at 490 nm.

### 9. Statistical Analysis

Statistical analysis was performed with GraphPad Prism 8 software, and all data were analyzed by t-test or one-way ANOVA (*p<0.05, *p<0.01, *p<0.001; ns means no significant differences). Experiment data were averaged by three independent experiments.

### Results:

1. Optimization of nABP284 and Synthesis of New Peptides
1.1 the Characteristic of nABP284

The amino acid sequence of nABP284 is SEQ ID NO: 7. The nABP284 of the present disclosure was synthesized by a solid-phase method and verified by ESI-MS/HPLC, as well as its purity. As shown in FIG. 3, Immunofluorescence technique was used for verifying whether nABP284 can specifically bind to PD-1 expressed on the surface of Jurkat T cells. In order to detect the binding affinity of nABP284 to re-combinant human PD-1 protein, surface plasma resonance (SPR) was used (FIG. 2A).

The nABP284 can specifically bind to PD-1 with moderate affinity. Flow cytometry showed that the binding rate between nABP284 and PD-1 on the stimulated Jurkat T cells may increase with its increasing concentration (FIG. 3).

1.2 Design of nABP386, nABP486 and nABP786

By analyzing the structure of PD-1 and its ligand PD-L1 (4ZQK) complex in RSCB PDB, homology of Human PD-L1 and nABP284 was compared. It was found that two regions, i.e. 2RLKEIA7 and 28Q in nABP284 are homologous to extracellular regions, i.e. 125RKYDA121 and 66Q in PD-L1, and they are just critical binding regions of PD-1 to PD-L1. Particularly, amino acid residues A121, K124, and R125 in the sequences are critical on the binding of PD-1 to PD-L1 (FIG. 1A and FIG. 1C). The region comprising residues A121, K124 and R125 is known as “Region 1”. In addition, amino acid sequence of SHHHRL was designed in a crack between Y68 and E136 of PD-1, where imidazole ring of histidine had a potential of binding to this crack. Structural analysis of computer modeling was performed with Autodock and Pymol, which indicated that the imidazole rings on three histidine of “SHHHRL” could bind to amino acid residues Region 1 of PD-1 to form hydrogen bond and obtain an enhanced binding (FIG. 1B). In conclusion, two new peptides, nABP386 and nABP486, are obtained by comparing homology between nABP284 and PD-L1 and adding the amino acid sequence comprising SHHHRL to the regions which may bind to PD-1 (FIG. 1C).

For further improving the binding affinity of nABP486 dual-targeting PD-1 and PD-L1, an amino acid sequence in backbone of nABP486 was optimized further to obtain a third new peptide, nABP786 (FIG. 1D).

1.3 Synthesis of nABP386, nABP486, and nABP786

nABP386, nABP486, and nABP786 were synthesized by China PolyPeptide Group Limited (Jiangsu) in a Fmoc solid-phase synthesis. The purity of these new peptides was analyzed and verified by ESI-MS/HPLC.

### 2. The Binding Specificity and Affinity of New Binding Peptides to PD-1

The binding affinities (Kd-value) of nABP386, nABP486, and nABP786 to PD-1 were detected by Surface plasma resonance (SPR), respectively. Recombinant Human PD-1 protein was coated on CM5 chip and the detected Kd-values of nABP386, nABP486, and nABP786 were 75.1 nmol/L, 11.9 nmol/L and 5.84 nmol/L, respectively (FIG. 2B, FIG. 2C and FIG. 2E), all of which were higher than the Kd-value of nABP284, 11.8 pmol/L (FIG. 2A).

Recombinant Human PD-L1 protein was coated on CM5 chip and the detected Kd-values of nABP486 and nABP786 were 52.4 nmol/L and 16.7 nmol/L, respectively, indicating that non-antibody binding peptides, nABP486 and nABP786 have a dual-target binding effect between PD-1 and PD-L1 (FIG. 2D).

When Jurkat cells were not stimulated by PMA, they hardly expressed PD-1. In contrast, when Jurkat cells were stimulated by PMA, they highly expressed PD-1 (FIG. 3). To prove the specific binding of new peptides nABP386, or nABP486 to PD-1, new peptides were used for binding to Jurkat T cells before and after stimulation, respectively. As shown in FIG. 3, nABP386 and nABP486 could bind to PD-1-expressing Jurkat T cells stimulated by PMA and ionomycin, but hardly bind to Jurkat T cells without stimulation. Compared with nABP284, the new peptides had a higher binding rate to PD-1-expressing Jurkat T cells, and the binding rate was positive correlation to the concentration (FIG. 4A and FIG. 4B). The results showed that the binding of nABP386 or nABP486 to PD-1-expressing Jurkat T cells, respectively, is a specific PD-1 mediated binding.

### 3. Blocking and Neutralizing Capacity of the New Peptides

The purpose of these studies was to determine whether the enhanced binding ability of new peptides corresponds to an improved blocking capacity or an improved neutralizing capacity, compared with nABP284. Jurkat T cells were stimulated with PMA and ionomycin for 24 hours. After the cells were incubated with nABP284, nABP386, nABP486, or functional anti-PD-1 antibody for 1 hour, respectively, the number of potential binding loci of PD-1 expression on the surface of Jurkat T cells that were incubated with peptides, was significantly reduced, compared with that of un-treated Jurkat cells (FIG. 5A). The quantitative results of mean fluorescence intensity indicated that nABP284, nABP386, nABP486 have a blocking capacity on PD-1 expressed on the surface of stimulated Jurkat T cells, and nABP486 has a stronger blocking capacity than nABP284 and nABP386 in an equal concentration, but there was no significance difference between nABP284 and nABP386 (FIG. 5B).

Further, neutralization capacity of nABP284 and new peptides to recombinant Human PD-L1 protein were studied. PD-L1, as a PD-1 ligand, can bind specifically to PD-1. nABP284, nABP386, or nABP486 was mixed with recombinant Human PD-L1 protein in a series of concentration, followed by binding to PD-1-expressing Jurkat T cells. The binding rate of recombinant Human PD-L1 protein was measured by flow cytometry, and the results indicated that the binding of recombinant Human PD-L1 protein to PD-1-expressing Jurkat T cells that were stimulated can be inhibited, by increasing the concentration of nABP284, nABP386, or nABP486 (FIG. 6D). The neutralization capacity of nABP386 and nABP486 are stronger than that of nABP284, and the neutralization capacity of nABP486 is strongest among the three peptides (FIG. 6A to FIG. 6D).

### 4. Inhibitory Effect of New Peptides Rescued Cal27 Cells on Jurkat T Cells

After Jurkat T cells were stimulated by PMA and ionomycin, PD-1 loci were highly expressed on the surface of the cells (FIG. 3), and Jurkat T cells secreted a large amount of IL-2. Similarly, the number of PD-L1 loci on the surface of the Cal27 cells was increased with the increased concentration of pre-stimulated IFN-γ (FIG. 7B). The secretion level of cytokines was an important indicator to evaluate T cell function. After co-incubation, Cal27 cells inhibited Jurkat T cells by the interaction of PD-1 with PD-L1, which was reflected in the fact that the secretion level of IL-2 detected by ELISA was decreased (FIG. 7D). In order to check whether the new peptides can more effectively inhibit the interaction of PD-1 with PD-L1, nABP284, nABP386, nABP486, or anti-PD-1 functional antibody was added to the culture system, after it was verified that nABP284, nABP386, nABP486 had no directly cytotoxicity to both Cal27 cells and Jurkat T cells. The results showed that the secretion level of IL-2 in the co-culture system was significantly increased after the addition of nABP284, nABP386, nABP486, or anti-PD-1 functional antibody, compared with the control group (FIG. 7D). Therefore, nABP284 and new peptides could recover the inhibition of Jurkat T cells by blocking the interaction of PD-1 with PD-L1, and nABP486 has the best blocking effect among the three peptides.

5. Effect of New Peptides: Enhanced iCIKs Killing-Efficiency on Tumor Cells

As shown in FIG. 7A, PBMCs were isolated from peripheral blood of donors, and then were induced by a series of cytokines comprising OKT-3, IL-2 and IFN-γ, and the induced PBMCs were named as cytokines induce killer cells (iCIKs). It was shown by flow cytometry that iCIKs significantly over-expressed PD-1 compared with the freshly isolated PBMCs (FIG. 7A). Cal27 cells stimulated by IFN-γ and iCIKs induced by cytokines were co-cultured and LDH level of the culture medium was measured by ELISA to evaluate iCIKs-mediated cytotoxicity for Cal27 cells. The iCIKs-mediated cytotoxicity for Cal27 cells was relatively weak (FIG. 7E), because there was a PD-1/PD-L1 pathway. When nABP486 as the most effective peptide in the above experiment was added into the co-culture system of iCIKs and Cal27 cells, the killing ability of iCIKs was significantly increased (FIG. 7E), compared with the control group. Considering that nABP486 had no direct killing ability to Cal27 cells (FIG. 7C), the above experimental results showed that nABP486 can block the interaction of PD-1 with PD-L1 and enhance iCIKs-mediated cytotoxicity (FIG. 7A and FIG. 7E).

In the study, it was proved that the two new peptides (nABP386 and nABP486) optimized from nABP284 in the previous studies were found to have a better effect on blocking the interaction of PD-1 with PD-L1. The binding affinity of PD-1 to nABP386 or nABP486 was significantly higher than that of nABP284. These peptides could block the binding of recombinant Human PD-L1 to PD-1 on the surface of stimulated Jurkat T cells. By adding such peptides into the co-culture system, the inhibitory effect of Cal27 cells on the Jurkat T cells and PBMCs can be reversed. In a word, the result showed that the peptides optimized from nABP284, in particular to, nABP486, had a great blocking effect on the interaction of PD-1 with PD-L1.

In the study, surface plasma resonance (SPR) results indicated that Kd-value of nABP386 binding to PD-1 was 75.1 nM, while Kd-value of nABP284 binding to PD-1 was 11.8 uM. Kd-value of nABP486 binding to PD-1 was 11.9 nM, while Kd-value of nABP486 binding to PD-L1 is 54.2 nM and nABP486 had a dual-target binding effect on PD-1 and PD-L1. Due to the dual-target blocking effect on PD-1 and PD-L1, it was hopeful to improve the inhibitory effect of tumor immunology checkpoints.

The above SPR results showed that after optimization of nABP284, the binding affinities of two peptides to PD-1 are higher than those to PD-L1, wherein the two peptides are optimized from nABP284. By comparing the PD-1 binding peptide sequences directly screened by phage display technology with the extracellular domain of PD-L1, the critical binding region of nABP284 was speculated and modified by adding histidine, which was formed as a side chain of nABP284, to the critical region of nABP284. Compared with nABP284, the binding affinity of the new peptide binding to PD-1 was significantly improved, which may be a more effective method to optimize the target binding peptide directly screened by phage display techniques.

The half effective inhibition concentration (IC50) of the peptides obtained from the neutralization experiments showed that the new peptides could effectively block PD-L1 from binding to PD-1 in a low concentration. A co-culture system, which was constituted of Jurkat cells and Cal27 cells, was taken as a model to simulate that PD-1 expressing tumor cells had an inhibitory effect on stimulated lymphocytes in vitro, and the inhibitory degree was demonstrated by evaluating the concentration level of IL-2. After the stimulated Jurkat cells were incubated with nABP386, nABP486 or functional anti-PD-1 antibody, the inhibition of stimulated Jurkat cells could be reversed by the interaction of PD-1 with PD-L1, and nABP486 had a more significant effect than anti-PD-1 antibody. PBMCs obtained from peripheral blood were cultured with cytokines such as anti-CD3 antibody, IL-2 and IFN-γ and the like for 10 days to obtain iCIK cells. Compared with PBMCs, iCIKs highly expressed PD-1 on their surfaces. When tongue cancer Cal27 cells were co-cultured with iCIKs, tongue cancer cells inhibited the tumor killing effect of iCIKs. When the stimulated iCIKs were pre-treated by nABP486 or functional anti-PD-1 antibody, they can significantly reverse the immune escape of tongue cancer cells to obtain an enhanced killing ability for tongue cancer cells. And the performance of nABP486 is superior to functional anti-PD-1 antibody. It is demonstrated by the study in vitro that the two new peptides (i.e. nABP386, nABP486) optimized from the PD-1 binding peptide, i.e. nABP284, have inhibitory effects on the interaction of PD-1 with PD-L1. Compared with nABP284, which was directly screened by phage display technology, nABP386 and nABP486 had better inhibitory effects in the same concentration, wherein nABP486 was superior to nABP386.

Important evaluation standards for an inhibitor of immune checkpoint include the stability in vivo, the half-life period and the binding affinity for target site. The addition of a side chain can generally increase the stability of the peptides in vivo. Compared with nABP284, both nABP386 and nABP486 have one side chain (SHHHRL), which can increase the stability of the new two peptides in vivo, thereby slowing the removal rate from metabolism.

The above mentioned examples are only embodiments of the invention and do not limit the invention in any form. Therefore, simple modification, equivalent change or modification of the above embodiments based on the technical essence of the invention shall still fall within the scope of the present invention, without deviating from the content of the invention.

Although the present invention has been disclosed in the form of preferred embodiments and variations thereon, it will be understood that numerous additional modifications and variations could be made thereto without departing from the scope of the invention.

For the sake of clarity, it is to be understood that the use of ‘a’ or ‘an’ throughout this application does not exclude a plurality, and ‘comprising’ does not exclude other steps or elements.

1. A non-antibody binding peptide or analogs thereof dual-targeting to a PD-1 and a PD-L1, wherein the non-antibody binding peptide comprises a backbone and at least one side chain linked to the backbone, wherein an amino acid sequence of the side chain is SEQ ID NO: 3, and an amino acid sequence of the backbone is at least one of SEQ ID NO: 1, SEQ ID NO: 2, and SEQ ID NO: 7.

2. A polynucleotide, encoding the non-antibody binding peptide of claim 1.

3. The polynucleotide of claim 2, wherein a nucleotide sequence encoding the backbone of the non-antibody binding peptide is SEQ ID NO: 4, and a nucleotide sequence encoding the side chain of the non-antibody binding peptide is SEQ ID NO: 6.

4. The polynucleotide of claim 2, wherein a nucleotide sequence encoding the backbone of the non-antibody binding peptide is SEQ ID NO: 5, and a nucleotide sequence encoding the side chain of the non-antibody binding peptide is SEQ ID NO: 6.

5. The polynucleotide of claim 2, wherein a nucleotide sequence encoding the backbone of the non-antibody binding peptide is SEQ ID NO: 8, and a nucleotide sequence encoding the side chain of the non-antibody binding peptide is SEQ ID NO: 6.

6. (canceled)

7. (canceled)

8. A preparation, comprising the non-antibody binding peptide or analogs thereof of claim 1, wherein the preparation is a drug or a cell marker reagent.

9. A diagnosis reagent for a tumor or an immune disease, the diagnosis reagent comprising the non-antibody binding peptide or analogs thereof of claim 1.

10. An immunoregulation drug or an anti-tumor drug, the immunoregulation drug or the anti-tumor drug comprising the non-antibody binding peptide analogs thereof of claim 1.
